(19) United States
(12) Patent Application Publication
(10) Pub. No.: US 2014/0277242 A1
(43) Pub. Date: Sep. 18, 2014

(54) MEDICAL DEVICE SYSTEM WITH ENERGY CONSUMPTION CALCULATION AND METHOD

(71) Applicant: MEDTRONIC, INC., Minneapolis, MN (US)

(72) Inventors: Sandra Beck Charlton, Little Rock, AR (US); James Jason Sims, Fayetteville, GA (US); Mark Richard Swanovich, Lino Lakes, MN (US)

(73) Assignee: MEDTRONIC, INC., Minneapolis, MN (US)

(21) Appl. No.: 13/829,792

(22) Filed: Mar. 14, 2013

(51) Int. Cl. A61N 1/37

(52) U.S. Cl. CPC A61N 1/3706
USPC 607/27; 607/2

(57) ABSTRACT

Implantable medical device and method provides pacing to a patient having a heart using a plurality of electrodes. Electrical circuitry is operatively coupled to each of the plurality of electrodes on each of the plurality of leads and is configured to provide a plurality of stimulation vectors with the plurality of electrodes to the heart of the patient. The electrical circuitry is configured to calculate an energy consumption for each of the plurality of stimulation vectors. The electrical circuitry is configured to take an action based, at least in part, on the energy consumption calculated for each of the plurality of stimulation vectors

### FIELD

The present invention relates generally to implantable medical devices and systems and related methods and, more particularly, to implantable medical devices and systems providing therapeutic stimulation to a patient.

### BACKGROUND

Implantable medical devices such as pacemakers are commonly configured to treat cardiac arrhythmias by delivering pacing pulses to cardiac tissue. Pacemakers commonly delivery therapy by way of electrodes positioned within or near the heart of the patient. Such therapy includes pacing therapy, which utilizes very low energy pulses designed to trigger cardiac contractions in lieu of an adequately frequent natural heart beat of the patient.

Pacemakers commonly incorporate a power source, such as a battery, which provides operational power to the componentry of the pacemaker, including electronics which manage the function of the device, monitor the condition of the patient in which the device is implanted and deliver therapy to the patient. Many or most device functions operate effectively continually, such as sensing the cardiac condition of the patient, or frequently, such as cardiac pacing therapy delivery in certain patients

Because pacemakers often provide life-sustaining therapy to the patients in which they are implanted and because pacemakers generally have a finite, limited energy resource, e.g., a battery, it is desirable to extend operation of the pacemaker by minimizing the energy used by the implantable medical device.

### SUMMARY

Such implantable medical devices, e.g., pacemakers, may deliver stimulation therapy, e.g., pacing therapy, over one or more stimulation vectors, e.g., pacing vectors, established between selected pairs of bipolar electrodes. The current across a vector between two stimulating electrodes uses electrical resources of the implantable medical device, usually a battery, creating an energy consumption on the electrical resources. Such energy or current is sometimes referred to as the “energy consumption” or “current drain”, i.e., the amount of energy or current, and resultant power, used to maintain the stimulation over a particular vector. Over time, such current drain can at least partially expend the capacity of the electrical resource, e.g., battery, of implantable medical device. Using the electrical resource of implantable medical device can, in turn, shorten the useful life of the battery and may require replacement of the battery which, in the case of an implantable medical device, can be a costly and inconvenient procedure.

In some situations, stimulating over a first vector using a first pair of electrodes may be efficacious as well as stimulating over a second vector using a second pair of electrodes may also be efficacious. However, one vector may have a higher resistance than the other and, hence, may result in a lower energy consumption. If both vectors are efficacious, it may be desirable to utilize the vector having a higher resistance, and lower energy consumption, in order to lower use of battery resources and increase the useful life of implantable medical device.

In an embodiment, an implantable medical device provides pacing to a patient, e.g., a patient having a heart, using a plurality of electrodes. Electrical circuitry is operatively coupled to each of the plurality of electrodes and is configured to provide a plurality of stimulation vectors with the plurality of electrodes to the patient, e.g., to the heart of the patient. The electrical circuitry is configured to calculate an energy consumption for each of the plurality of stimulation vectors. The electrical circuitry is configured to take an action based, at least in part, on the energy consumption calculated for each of the plurality of stimulation vectors.

In an embodiment, each of the plurality of leads is operatively coupled to a plurality of electrodes.

In an embodiment, at least one of the plurality of electrodes comprises a left ventricular pacing electrode and wherein the energy consumption calculated by the electrical circuitry comprises a left ventricular energy consumption.

In an embodiment, the electrical circuitry is configured to calculate a energy consumption for each of the plurality of stimulation vectors that achieves a predetermined efficacy.

In an embodiment, the electrical circuitry is configured with action comprising displaying, by each of the plurality of stimulation vectors, to a user the energy consumption calculated by the electrical circuitry.

In an embodiment, the electrical circuitry is configured wherein energy consumption is displayed only for ones of the plurality of stimulation vectors achieving a predetermined efficacy.

In an embodiment, the electrical circuitry is configured with the displaying ranking the energy consumption for each of the plurality of stimulation vectors based, at least in part, on a value of the energy consumption.

In an embodiment, the electrical circuitry is configured to automatically select ones of the plurality of stimulation vectors based, at least in part, on the energy consumption and the efficacy of each of the plurality of vectors.

In an embodiment, the electrical circuitry is configured to automatically select ones of the plurality of stimulation vectors based, at least in part, having a lower value of the energy consumption while achieving a minimum of the predetermined efficacy.

In an embodiment, the electrical circuitry is configured to calculate the energy consumption for each of the plurality of stimulation vectors by (1) ramping up an amplitude of one of the plurality of stimulation vectors until capture occurs.

In an embodiment, the electrical circuitry is further configured to calculate the energy consumption for each of the plurality of stimulation vectors by (2) reducing the amplitude of the one of the plurality of stimulation vectors until loss of capture occurs, and (3) returning the amplitude to the amplitude at which capture previously occurred.

In an embodiment, the electrical circuitry is further configured to calculate the energy consumption for each of the plurality of stimulation vectors by (2) reducing the amplitude of the one of the plurality of stimulation vectors until loss of capture occurs, and (3) again increasing the amplitude the amplitude until capture occurs.

In an embodiment, a device-implemented method for using an implantable medical device uses a plurality of electrodes. A plurality of stimulation vectors is delivered with the plurality of electrodes to a patient, e.g., a heart of a patient. An energy consumption is calculated for each of the plurality of stimulation vectors. An action is taken based, at least in part, on the energy consumption calculated for each of the plurality of stimulation vectors.

In an embodiment, a plurality of stimulation vectors is delivered with the plurality of electrodes to a heart of a patient, each of the plurality of electrodes operatively coupled to a plurality of leads.

In an embodiment, at least one of the plurality of electrodes comprises a left ventricular pacing electrode and wherein the calculating step is accomplished by calculating a left ventricular energy consumption.

In an embodiment, a energy consumption is calculated for each of the plurality of stimulation vectors that achieves a predetermined efficacy.

In an embodiment, the energy consumption for each of the plurality of stimulation vectors is displayed to a user.

In an embodiment, the energy consumption is displayed only for ones of the plurality of stimulation vectors achieving a predetermined efficacy.

In an embodiment, the energy consumption is displayed for each of the plurality of stimulation vectors based, at least in part, on a value of the energy consumption.

In an embodiment, ones of the plurality of stimulation vectors are automatically selected based, at least in part, on the energy consumption and the efficacy of each of the plurality of vectors.

In an embodiment, ones of the plurality of stimulation vectors are automatically based, at least in part, having a lower value of the energy consumption while achieving a minimum of the predetermined efficacy.

In an embodiment, the energy consumption for each of the plurality of stimulation vectors is calculated by (1) ramping up an amplitude of one of the plurality of stimulation vectors until capture occurs.

In an embodiment, the energy consumption for each of the plurality of stimulation vectors is calculated by (2) reducing the amplitude of the one of the plurality of stimulation vectors until loss of capture occurs, and (3) returning the amplitude the amplitude to the amplitude at which capture previously occurred.

In an embodiment, the energy consumption for each of the plurality of stimulation vectors is further calculated by (2) reducing the amplitude of the one of the plurality of stimulation vectors until loss of capture occurs, and (3) again increasing the amplitude the amplitude until capture occurs.

### FIGURES

FIG. 1 is a schematic block diagram of an implantable medical device therapeutically coupled to the heart of a patient;

FIG. 2 is an embodiment of a display displaying a ranked order of calculated current drains by vector;

FIG. 3 is an alternative embodiment of a display displaying a ranked order of calculated current drains for only certain vectors; and

FIG. 4 is a flow chart of an embodiment of a method of using an implanted medical device utilizing calculated current drains.

### DESCRIPTION

FIG. 1 is an illustration of implantable medical device 10. In the illustrated embodiment, implantable medical device 10 is a cardiac pacemaker. The pacing function may treat bradycardia and may resynchronize heart 12 in conditions of patient heart failure. Implantable medical device 10 could also be a cardiac resynchronization therapy defibrillator, known in the art as a CRT-D device. Implantable medical device 10 is coupled to heart 12 by way of left ventricular lead 14 and right ventricular lead 16. In other embodiments, implantable medical device 10 may utilize solely left ventricular lead 14, or utilize a coronary sinus lead, left atrial lead or a right atrial lead, in addition to or instead of left ventricular lead 14.

As illustrated, right ventricular lead 16 is positioned such that its distal end is in the right ventricle for sensing right ventricular cardiac signals and delivering pacing or shocking pulses in the right ventricle. Left ventricular lead 14 is positioned such that its distal end is in the left ventricle for sensing left ventricular cardiac signals and delivering pacing or shocking pulses in the left ventricle.

Left ventricular lead 14 and right ventricular lead 16 are operatively coupled to output section 18 of implantable medical device 10. Output section 18 is conventional and supplies appropriate electrical stimulation to heart 12 and may receive sensing information from heart to aid in therapeutically stimulating heart 12. Output section 18 is controlled by control section 20 of implantable medical device 10.

Left ventricular lead 14 contains electrodes 22, 24, 26 which are disposed in or around locations of the left ventricle of heart 12. Electrodes 22, 24, 26 may be individually controlled by output section 18. Similarly, right ventricular lead 16 contains electrodes 28, 30, 32 which are disposed in or around locations of the right ventricle of heart 12. Electrodes 28, 30, 32 may also be individually controlled by output section 18. For purposes of sensing information from heart 12 and for purposes of actually supplying therapeutic stimulation to heart 12, left ventricular lead 14 and electrodes 22, 24, 26 as well as right ventricular lead 16 and electrodes 28, 30, 32 are operated conventionally in accordance with well known implantable medical device principles.

It is to be recognized and understood that while electrodes 22, 24 and 26 and electrodes 28, 30 and 32 are illustrated as being positioned in the ventricles of heart 12 that other conventional heart locations for electrodes are also contemplated. For example, some or all of electrodes 22, 24, 26, 28, 30, 32 may be positioned in or around other portions of heart 12 such as the atria, the right atria and the coronary sinus. It is also to be recognized and understood that electrodes 22, 24, 26, 28, 30, 32 may also be positioned outside of heart 12, e.g., subcutaneously, to sense information from and/or provide stimulation to heart 12.

In each case, output section 18 of implantable medical device 10 may operate electrodes 22, 24, 26, 28, 30, 32 in a bipolar paired arrangement, establishing a sensing and/or stimulation “vector” between a pair of electrodes. For example, utilizing electrodes 22 and 26 in the left ventricle establishes vector 34. Utilizing electrodes 22 and 24 in the left ventricle establishes vector 36. Utilizing electrodes 28 and 32 in the right ventricle establishes vector 38. Utilizing electrodes 28 and 30 in the right ventricle establishes vector 40 and utilizing electrodes 30 and 32 in the right ventricle establishes vector 42.

Various ones of such bipolar pairs may be referred to as a “tip-to-ring” pairs. Electrodes 22, 24, 26, 28, 30, 32 may likewise be utilized individually in unipolar configuration with the housing of implantable medical device 10 serving as an indifferent electrode, commonly referred to as the “can” or “case” electrode. In various embodiments, alternate lead systems may be substituted for the lead system of the embodiment of FIG. 1. Leads for use with single chamber, dual chamber, or multi-chamber implantable medical devices may be utilized.

Output section 18 provides selected electrode pairs, e.g., bipolar electrode pairs, with a stimulating signal. In certain embodiments, a voltage or a signal with a varying voltage having an amplitude is applied across an electrode pair, e.g., electrodes 22 and 26, establishing a voltage across vector 34. The amount, or amplitude, of the current flowing between electrodes 22 and 26 across vector 34 may be largely dependent upon the resistance of tissue and/or fluid encountered by vector 34. Even if a voltage or signal with a voltage of equal amplitude is placed across different vectors, the current can be expected to vary based, in large part, on the resistance encountered. A vector with a relatively high resistance can result in a relatively low current across the vector. Conversely, a vector with a relatively low resistance can result in a relatively high current across the vector.

The current across a vector between two stimulating electrodes uses electrical resources of implantable medical device 10, usually a battery. Such current is sometimes referred to as the “energy consumption” or “current drain”, i.e., the amount of current or energy, and resultant power, used to maintain the stimulation over a particular vector. Over time, such energy consumption or current drain can at least partially expend the capacity of the electrical resource, e.g., battery, of implantable medical device 10. Using the electrical resource of implantable medical device can, in turn, shorten the useful life of the battery and may require replacement of the battery which, in the case of an implantable medical device, can be a costly and inconvenient procedure.

Often, multiple electrodes are implanted as illustrated in FIG. 1. Also as illustrated in FIG. 1, a plurality of vectors may be used for stimulation using selected pairs of the multiple electrodes. In some situations, stimulating over a first vector using a first pair of electrodes may be efficacious as well as stimulating over a second vector using a second pair of electrodes may also be efficacious. However, one vector may have a higher resistance than the other and, hence, may result in a lower current drain. If both vectors are efficacious, it may be desirable to utilize the vector having a higher resistance, and lower current drain, in order to lower use of battery resources and increase the useful life of implantable medical device 10.

Upon implantation of implantable medical device 10 and electrodes 22, 24, 26, 28, 30, 32, it may not be known what the impedance, or resistance, between selected electrode pairs, and hence what the impedance, or resistance, of each vector will be when implantable medical device is operational. Further, the impedance of each vector may vary over time, e.g., due to changes in fluid in the path of the vector. Therefore, the clinician or programmer of implantable medical device may not know which vector or vectors and, hence, which electrode pairs will have a lower or higher current drain. Selected an electrode pair having a lower resistance could result in a vector having a higher current drain and a shortened battery life unbeknownst to the clinician.

Control section 20 may be configured drive, e.g., sequentially drive, available multiple vectors established by selected pairs of electrodes, to test each vector and determine the relative current drain for each vector based on a given stimulation voltage. For example, in an in-office patient session, an energy usage calculation may be run sequentially or simultaneously on each vector during an automatic threshold test.

Once the current drain of each of the plurality of vectors is established, control section may take an action based, at least in part, of the results of the current drain testing.

In an embodiment, control section 20 may display (FIG. 2) the current drain, or some indicia representation of current drain, using display 44. In an embodiment, display 44 is located on clinician equipment in the office setting. In general, however, display 44 may be any mechanism for displaying the information obtained from implantable medical device, for example by conventional telemetry, and could be located local to the office session, e.g., on a physician programmer, or could be displayed remotely through any of many commonly known data links.

In the embodiment illustrated in FIG. 2, display 44 shows the current drain 44 of each vector 48 in ranked 50 order. For example, the top rated rank 50 is the vector having the lower current drain 46. Remaining vectors are displayed in order of increasing current drain 46. By displaying relative current drain, it is easy for the clinician to determine which vector or vector have a lower or the lowest current drain 46 and allows the clinician to select a vector (and the electrode pair establishing that vector) having the most appropriate current drain 46 while maintaining whatever additional criteria the clinician may take into consideration.

It is not necessary that display 44 actually display the rank 50 number. For example, simply displaying the vectors in order of increasing, or decreasing, current drain 46 allows the clinician to easily determine the vector or vectors with the higher or lower current drains 46.

It is also not necessary to display the actual current drain 46 for each vector or for any vector. Again, simply displaying the vectors in order of increasing, or decreasing, current drain 46 allows the clinician to easily determine the vector or vectors with the higher or lower current drains 46 without necessarily having reference to the actual current drain.

In an embodiment, display 44 also displays, for each vector, the efficacy level or, as illustrated, generally whether or not each particular vector achieves a particular efficacy 52. For example, even though a particular vector may have a very low current drain 46, such vector may have the efficacy 52 desired by the clinician. For example, in the case of a pacemaker, a particular vector may not be able to achieve, or consistently achieve, capture during pacing operation. Displaying efficacy in display 44 can allow the clinician to disregard or eliminate from consideration one or more vectors that, although having the beneficial result of low current drain 46, however, are not desirable because of lack of or lower efficacy 52.

In an embodiment, display 44, instead of displaying a ranking of each vector, may display a ranking of only vectors which have achieved the desired or specified, e.g., predetermined level of, efficacy. This is illustrated in FIG. 3 in which current drain 46 is displayed for vectors 3, 6, 5, 2 and 4. As noted in FIG. 2, vector 1 did not achieve the specified efficacy and, hence, has been omitted from display 44 in FIG. 3.

It may also be desirable for the clinician to otherwise specify certain vector or vectors to be omitted from display 44. For example, a vector which satisfies the efficacy specification may nonetheless be undesirable because, for example, of also producing undesirable side effects. In such case, the clinician could de-select certain vectors or groups of vectors, certain electrodes or groups of electrodes or certain leads or groups of leads. Display 44 would display only vectors that are de-selected from consideration. Of course, it is to be recognized and understood that the clinician could positively select certain vectors or groups of vectors, certain electrodes or groups of electrodes or certain leads or groups of leads for inclusion in display 44 instead of de-selecting as indicated above. It is also to be recognized and understood that certain vectors or groups of vectors, certain electrodes or groups of electrodes or certain leads or groups of leads could be omitted from display 44 by automatic analysis instead of manual identification by a clinician.

In an embodiment, the clinician could choose the optimal stimulation vector, e.g., a pacing vector, such as a left ventricular pacing vector, based on the data provided and the clinician's clinical knowledge of the patient or even ignore the recommendation. For instance, if the optimal energy saving vector also produces phrenic nerve stimulation or unsuitable hemodynamics, it could be disqualified by the clinician or programmed off, thus saving future implantable medical device 10 energy consumption and memory storage of the results.

In an embodiment, control section 20 may automatically select a vector or a plurality of vectors based, at least in part, on the results of the current drain 46 calculation operating as a closed loop system. The automatic energy calculations could be run of each viable vector and the pacing vector could be automatically changed by control section 20 to the vector achieving the lowest current drain 46, or the vector with the lowest current drain 44 which still achieves predetermined efficacy 52 or which met preset safety margins. Optimization of battery longevity could be achieved with remote monitoring instead of relying on in-office patient sessions. The same automatic energy calculation for all vectors, or all left ventricular vectors, could be utilized from a mobile application for a “smart device” that could be utilized by technical or clinical support, or others, to improve time efficiency.

In an embodiment, efficacy of each vector may be determined by calculating current drain 46 for each vector by ramping up the amplitude of each vector until pacing capture occurs. This value or a related value, first amplitude achieving capture or an amplitude greater than first amplitude to achieve a safety margin, may be used for the calculated current drain. Further, the amplitude may then be reduced until loss of capture occurs and again increased in amplitude until capture re-occurs.

While control section 20 has been illustrated as being part of implantable medical device 10, it is to be recognized and understood that in certain in-office clinical environments, some or all of current drain 46 calculations could be off loaded to external equipment in or operatively coupled to the clinical environment of implantable medical device 10 in order to reduce the computational load on implantable medical device 10.

In FIG. 4, a method of using implantable medical device 10 is illustrated. Implantable medical device 10 is utilized to deliver (410) a plurality of pacing vectors with electrodes 22, 24, 26, 28, 30, 32. The current drain 46 of each vector is calculated (412) for each of the plurality of vectors. An action is taken (414) based, at least in part, on the calculated current drain 46. The action taken (414) may comprise displaying (416) the results of current drain 46 for each vector 48. Such display may be in ranked order. In an embodiment, current drains 46 or left ventricular current drains. In an embodiment, only vectors achieving a predetermined efficacy are displayed. In an embodiment, the action taken (414) may comprise automatically selecting (418) one or more vectors based, at least in part, on the calculated current drain 46.

Thus, embodiments of the medical device system with energy consumption calculation and method are disclosed. One skilled in the art will appreciate that the present invention can be practiced with embodiments other than those disclosed. The disclosed embodiments are presented for purposes of illustration and not limitation, and the present invention is limited only by the claims that follow.

What is claimed is:

1. A medical system, comprising:
an implantable medical device for providing electrical stimulation to a patient, said implantable medical device having:
a plurality of electrodes; and
electrical circuitry, operatively coupled to each of said plurality of electrodes, being configured to provide a plurality of stimulation vectors with said plurality of electrodes to said patient; and
said electrical circuitry being configured to calculate an energy consumption for each of said plurality of stimulation vectors;
said medical system being configured to take an action based, at least in part, on said energy consumption calculated for each of said plurality of stimulation vectors.

2. The medical system of claim 1 further comprising a plurality of leads wherein each of said plurality of leads are operatively coupled to a plurality of electrodes.

3. The medical system of claim 2 wherein at least one of said plurality of electrodes comprises a left ventricular pacing electrode and wherein said energy consumption calculated by said electrical circuitry comprises a left ventricular energy consumption.

4. The medical system of claim 3 wherein said electrical circuitry is configured to calculate an energy consumption for each of said plurality of stimulation vectors that achieves a predetermined efficacy.

5. The medical system of claim 3 wherein said electrical circuitry is configured with action comprising displaying, by each of said plurality of stimulation vectors, to a user said energy consumption calculated by said electrical circuitry.

6. The medical system of claim 5 wherein said electrical circuitry is configured wherein energy consumption is displayed only for ones of said plurality of stimulation vectors achieving a predetermined efficacy.

7. The medical system of claim 6 wherein said electrical circuitry is configured with said displaying ranking said energy consumption for each of said plurality of stimulation vectors based, at least in part, on a value of said energy consumption.

8. The medical system of claim 3 wherein said electrical circuitry is configured to automatically select ones of said plurality of stimulation vectors based, at least in part, on said energy consumption and said efficacy of each of said plurality of vectors.

9. The medical system of claim 8 wherein said electrical circuitry is configured to automatically select ones of said plurality of stimulation vectors based, at least in part, having a lower value of said energy consumption while achieving a minimum of said predetermined efficacy.

10. The medical system of claim 1 wherein said electrical circuitry is configured to calculate said energy consumption for each of said plurality of stimulation vectors by (1) ramping up a magnitude of one of said plurality of stimulation vectors until capture occurs.

11. The medical system of claim 10 wherein said electrical circuitry is further configured to calculate said energy consumption for each of said plurality of stimulation vectors by (2) reducing said amplitude of said one of said plurality of stimulation vectors until loss of capture occurs, and (3) returning said amplitude said amplitude to said amplitude at which capture previously occurred.

12. The medical system of claim 10 wherein said electrical circuitry is further configured to calculate said energy consumption for each of said plurality of stimulation vectors by (2) reducing said amplitude of said one of said plurality of stimulation vectors until loss of capture occurs, and (3) again increasing said amplitude said amplitude until capture occurs.

13. A device-implemented method using an implantable medical device having a plurality of electrodes, comprising the steps of:
delivering a plurality of stimulation vectors with said plurality of electrodes to a heart of a patient;
calculating a energy consumption for each of said plurality of stimulation vectors; and
taking an action based, at least in part, on said energy consumption calculated for each of said plurality of stimulation vectors.

14. The method of claim 13 wherein said delivering step further comprises delivering a plurality of stimulation vectors with said plurality of electrodes to a heart of a patient, each of said plurality of electrodes operatively coupled to a plurality of leads.

15. The method of claim 14 wherein at least one of said plurality of electrodes comprises a left ventricular pacing electrode and wherein said calculating step is accomplished by calculating a left ventricular energy consumption.

16. The method of claim 15 wherein said calculating step is accomplished by calculating a energy consumption for each of said plurality of stimulation vectors that achieves a predetermined efficacy.

17. The method of claim 15 wherein said taking an action step comprises displaying, by each of said plurality of stimulation vectors, to a user said energy consumption calculated by said electrical circuitry.

18. The method of claim 16 wherein said displaying step comprises displaying said energy consumption only for ones of said plurality of stimulation vectors achieving a predetermined efficacy.

19. The method of claim 18 wherein said displaying step comprises displaying said energy consumption for each of said plurality of stimulation vectors based, at least in part, on a value of said energy consumption.

20. The method of claim 15 wherein said taking an action step comprises automatically selecting ones of said plurality of stimulation vectors based, at least in part, on said energy consumption and said efficacy of each of said plurality of vectors.

21. The method of claim 20 wherein said selecting step comprises automatically selecting ones of said plurality of stimulation vectors based, at least in part, having a lower value of said energy consumption while achieving a minimum of said predetermined efficacy.

22. The method of claim 13 wherein said calculating step comprises calculating said energy consumption for each of said plurality of stimulation vectors by (1) ramping up an amplitude of one of said plurality of stimulation vectors until capture occurs.

23. The method of claim 22 wherein said calculating step comprises calculating said energy consumption for each of said plurality of stimulation vectors by (2) reducing said amplitude of said one of said plurality of stimulation vectors until loss of capture occurs, and (3) returning said amplitude said amplitude to said amplitude at which capture previously occurred.

24. The method of claim 22 wherein said calculating step comprises calculating said energy consumption for each of said plurality of stimulation vectors by (2) reducing said amplitude of said one of said plurality of stimulation vectors until loss of capture occurs, and (3) again increasing said amplitude said amplitude until capture occurs.
